(19) United States
(12) Patent Application Publication
(10) Pub. No.: US 2015/0022093 A1
(43) Pub. Date: Jan. 22, 2015

(54) LED LIGHT CONTROLLER AND METHOD OF CONTROLLING LED LIGHTS

(71) Applicant: USaveLED, Boca Raton, FL (US)

(72) Inventors: Rodney G. Smith, Boca Raton, FL (US); Harry Zuker, Delray Beach, FL (US)

(21) Appl. No.: 14/337,894

(22) Filed: Jul. 22, 2014

Related U.S. Application Data
(60) Provisional application No. 61/856,924, filed on Jul. 22, 2013.

(51) Int. Cl. H05B 33/08; A61M 21/02; H05B 37/02; A61N 5/06

(52) U.S. Cl. CPC H05B 33/0854; A61N 5/0618; A61M 21/02; H05B 37/0218; A61M 2021/0044
USPC 315/151; 315/307

(57) ABSTRACT

A method controls the light exposure of an individual during a given time period. A control unit is provided for controlling lights. A first sensor is worn by the individual and gathers light exposure data including lighting intensity data and Kelvin temperature data experienced by the individual. Second sensors are disposed in a building for collecting emitted light data emitted in the building. The emitted light data and the light exposure data are transmitted to the control unit. The light data, along with desired data including desired light intensity and desired Kelvin temperature are stored. The optimal light exposure for the individual is determined based on the light data or the desired data, and an output signal is generated based on the optimal light exposure. The lights are controlled based on the output signal to produce an overall light intensity and Kelvin temperature pattern per day for the individual.

### CROSS-REFERENCE TO RELATED APPLICATION

This application claims the priority, under 35 U.S.C. §119, of U.S. provisional patent application No. 61/856,924, filed Jul. 22, 2013; the prior application is herewith incorporated by reference in its entirety.

### BACKGROUND OF THE INVENTION

### Field of the Invention

The present invention relates to LED lighting control. More particularly, it relates to control of Kelvin temperature and light intensity of LED lighting in order to adapt to human lighting needs in a way to overcome deficiencies of lighting spectrum exposure commonly found with traditional artificial lighting, and applications thereof, with sensor feedback through intelligent control mechanisms.

Lighting and devices to control lighting are vital to modern society and profoundly affect normal brain function through effecting or preventing secretion of chemicals by the pineal gland in the brain, as well as cortisol secreted by the adrenal gland, and several other hormones including dopamine. Widespread use of artificial lighting has been shown to disrupt sleep patterns especially among the young and old, as well as shift workers. Lighting control has been primarily focused on delivering the desired quantity of light with little consideration as to optimal levels of Kelvin temperature of the lighting through normal daily cycles. This trend has resulted in record numbers of people requiring pharmaceutical sleep aids and other interventions.

The advent of artificial lighting during the late 19^{th} century and widespread deployment during the 20^{th} century has resulted in disruption of normal light exposure patterns, e.g. exposure to bright-white sunlight in the morning (high Kelvin temperature), and diminished Kelvin temperature at sundown that is essential to trigger normal sleep cycles among other behaviors. What is needed is a Kelvin variable light, similar in capability to a dimmable light whereby the quantity of light is adjusted to suit the activity, whereby individuals and groups can be subjected to desirable Kelvin temperatures of light for specific activities. On a broader scale, e.g. an old age facility, hospital, or school, it would be particularly useful if the Kelvin variable LED light fixtures could be controlled through an integrated wireless control system. Furthermore, it would be even more advantageous if data gathering sensors could be used to provide feedback regarding light exposure to an intelligent control module whereby the Kelvin temperature and intensity of light could be automatically controlled to adapt and optimize light levels sensitive to human behavior goals.

### BRIEF SUMMARY OF THE INVENTION

The present adaptive lighting invention provides an LED flat panel luminaire or other LED lighting format that includes the abilities to both be controlled through dimming and Kelvin variability, and to provide programmable scheduling of dimming and Kelvin temperature control among other control features. A large number of such lights can be controlled through wired or wireless radio frequency (RF) control systems whereby individual lights, groups of lights, or all the lights can be programmatically controlled. Furthermore, the lighting controller can accept input from sensors, including but not limited to ambient lighting conditions by space, group, and individual; can process that input along with certain control orders or policies established in the control system, and effect intelligent control of the lighting devices consistent with defined human needs including but not limited to requirements of circadian rhythm. While it has been shown that through active light therapy involving exposure to defined levels of Kelvin temperature human behavior can be modified, our invention involving widespread deployment of Kelvin variable lighting integrated with intelligent controls can passively achieve desired Kelvin temperatures and intensity to create a healthier lighting environment for individuals and groups, while automating the adaptive lighting controls.

It is a feature of our system that lighting environments can be automatically controlled for Kelvin temperature in pre-programmed manners such as scheduling specific control actions, as well as through feedback from integrated data gathering sensors whose output is automatically processed and used as input to the controller. An important capability of the control system is its ability to resolve control conflicts that may occur by different manual and automated control commands. The control module provides the ability to schedule future lighting control actions, certain control settings for emergency situations, and individual control commands. To assure conflict resolution between control commands the control module has a functionality composer that evaluates the user class and task for manual or scheduled control, and certain automated controls in relation to each other in a hierarchical manner to determine which control actions may override other control actions.

Furthermore certain emergency situations are definable that may override most if not all other control commands. For example when the invention is deployed in a K-12 school environment a policy could be established that would use the lighting system to provide visual warning of dangerous situations such as a gunman being loose on the campus. The processing of this lighting control command would override other commands such as dimming the lights to facilitate display of video media in class. Likewise a “code blue” condition of a patient in a hospital patient room would alert the lighting command module of the status and a lighting control command would be issued to provide a predefined level of bright light, e.g. 100 foot candles, immediately over the patient bed. Other lighting commands would not be processed until the code blue is cleared unless approved by an authorized individual such as a doctor.

With the foregoing and other objects in view there is provided, in accordance with the invention, a method for controlling light exposure of an individual. The method includes providing a control unit controlling individual and groups of lights controlled by a power switch, the control unit having a memory unit. A first sensor is worn by the individual for gathering individual light data including lighting intensity data and Kelvin temperature data experienced by the individual. The first sensor transmits the individual light data to the control unit. Second sensors are disposed at various locations in a building used by the individual for collecting building light data including light intensity building data and Kelvin temperature building data emitted in the building. The building light data is also transmitted to the control unit. The memory unit of the control unit stores the individual light data and the building light data, along with desired data including desired light intensity and desired Kelvin temperature. An optimal light exposure for the individual or the individual within a group based on at least one of the individual light data, the building light data or the desired data is determined. At least one output signal based on the optimal light exposure is generated. The at least one output signal is sent to the control unit. At least one of the power switch, a light dimming switch or a Kelvin temperature changing switch controls the lights based on the output signal to produce an overall light intensity and Kelvin temperature pattern for the individual. In this manner, one combines the known exposed light already received by the individual with a desired amount of light exposure and determines how much more light the individual must receive for being exposed for the desired amount.

In accordance with an added mode of the invention, a fuzzy neural network processing unit is used for determining the optimal light exposure for the individual based on at least one of the individual light data, the building light data or the desired data.

In accordance with another mode of the invention, the method weights the inputs to the fuzzy neural network processing unit for optimizing the light exposure needs of an individual within a group such that the individual in the group most in need of light intensity and Kelvin temperature optimization is given a greater weighting within the group in determining an optimal light intensity and the Kelvin temperature for the group.

In this manner, the individual who has a light exposure pattern farthest from the desired light exposure is given the greatest weighting for determine the light exposure a group is to receive. This is only possible because of the electronic tracking of each individual within the group.

In accordance with a further mode of the invention, activity data stored in the memory unit relating to planned activities in advance of a particular activity are used by the control unit to optimize the light intensity and the Kelvin temperature for the individual in such a way as to deliver light consistent with scientific studies that indicate that behavior is influenced in a desired manner when the individual is exposed to specific levels of light intensity and the Kelvin temperature. The planned activity can be sleep patterns, testing taking periods, activities performed in mornings and activities performed in evening hours which all required customized lighting needs.

In accordance with an additional mode of the invention, the quantity of the light delivered to the individual on a daily basis is based on a 24 hour circadian rhythm and the light based on the circadian rhythm is adjusted or reviewed at least once per hour.

Other features which are considered as characteristic for the invention are set forth in the appended claims.

Although the invention is illustrated and described herein as embodied in an LED light controller and a method of controlling the LED lights, it is nevertheless, not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF SUMMARY OF THE SEVERAL VIEWS OF THE DRAWINGS

FIG. 1 is a diagrammatic illustration of a WalaLight LED lighting system including data gathering sensors, telecommunications, control systems, and LED lighting according to the invention;

FIG. 2 is a perspective view of a WalaLight light intensity and Kelvin temperature data gathering pin with radio frequency telecommunications capability;

FIG. 3 is an illustration of building and area lighting sensors;

FIG. 4 is a block diagram of main components of a central control unit depicting data gathering, data analysis, prediction, and control components;

FIG. 5 is a block diagram of a central control unit prediction module fuzzy neural network;

FIG. 6 is a block diagram of the training element of the WalaLight prediction module;

FIG. 7 is an illustration of a WalaLight programmable controller interface;

FIG. 8 is an illustration of a wireless radio frequency WalaLight hand held controller with light intensity and Kelvin temperature display and programmable buttons;

FIG. 9 is an illustration of a wired/wireless radio frequency WalaLight wall controller;

FIG. 10 is an illustration of programmable WalaLight controller for use in a senior care facility;

FIG. 11 is an illustration of programmable WalaLight configurations in an educational facility;

FIG. 12 is an illustration of a WalaLight integrated with intelligent building control systems;

FIG. 13 is an illustration of a programmable WalaLight home kitchen fixture;

FIG. 14 is an illustration of programmable WalaLight home bathroom fixture;

FIG. 15 is an illustration of a WalaLight LED flat panel; and

FIG. 16 is an illustration of a control module.

### DETAILED DESCRIPTION OF INVENTION

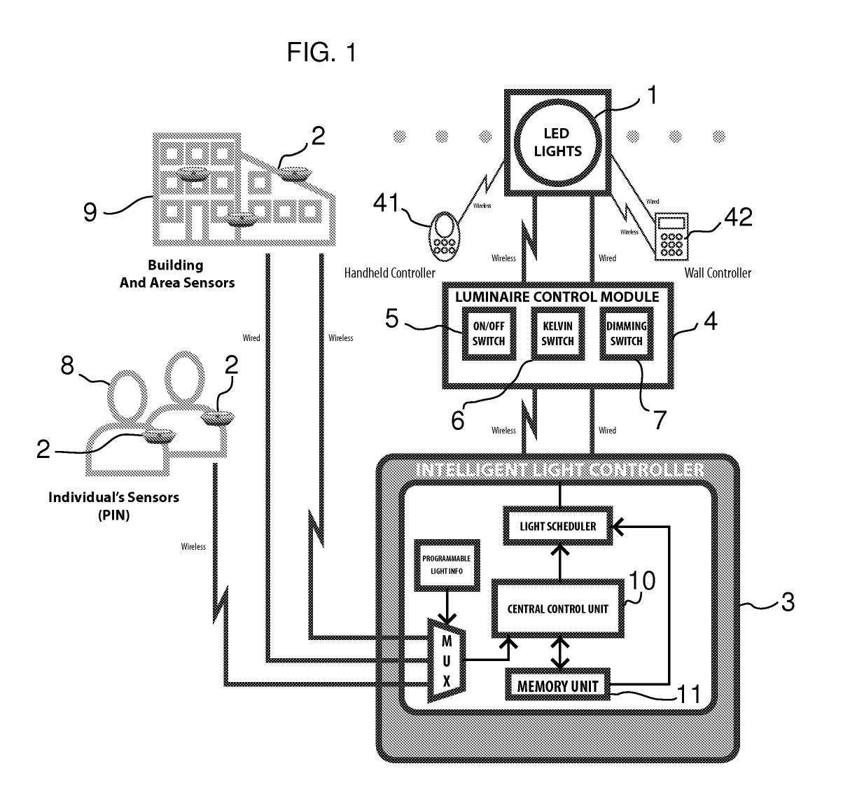

Referring now to the figures of the drawings in detail and first, particularly to FIG. 1 thereof, there is shown an integrated system containing a dimmable and Kelvin temperature changeable LED light unit or panel 1, a variety of light sensors 2, and a lighting control system 3 that can be pre-programmed, e.g. by schedule; and can also change lighting intensity and/or Kelvin temperature from sensor feedback that is automatically processed in the lighting control system 3 and issues control commands to the LED light unit 1 that is configured to optimize exposure to light for individuals and groups so as to brighten or dim the lights 1, and/or change the Kelvin temperature from warm white (2,700 Kelvin) to cool white (6,500 Kelvin). The LED light unit 1 is directly controlled by a luminaire control module 4 having an on/off switch 5, a Kelvin temperature control switch 6 and a dimming switch 7. The luminaire control modules 4 throughout a facility are controlled by the master lighting control system 3. The luminaire control modules 4 may be in the form of a wall controller 42 or a handheld controller 41.

As further shown in FIG. 1, the light sensors 2 can be individual light sensors physically carried by an individual 8 or stationed throughout a building 9 at various locations.

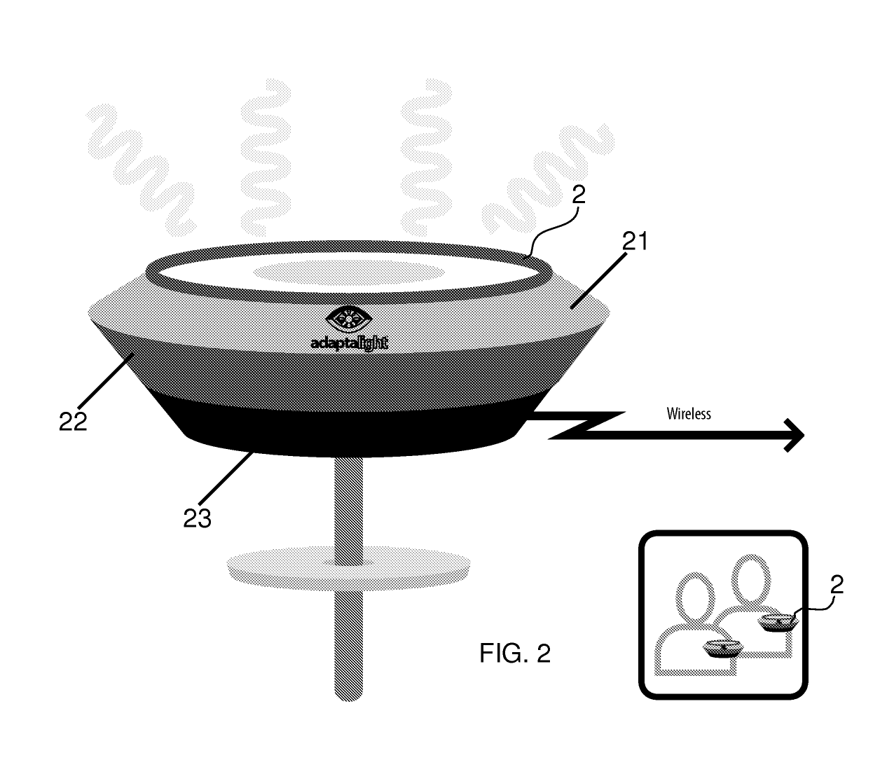

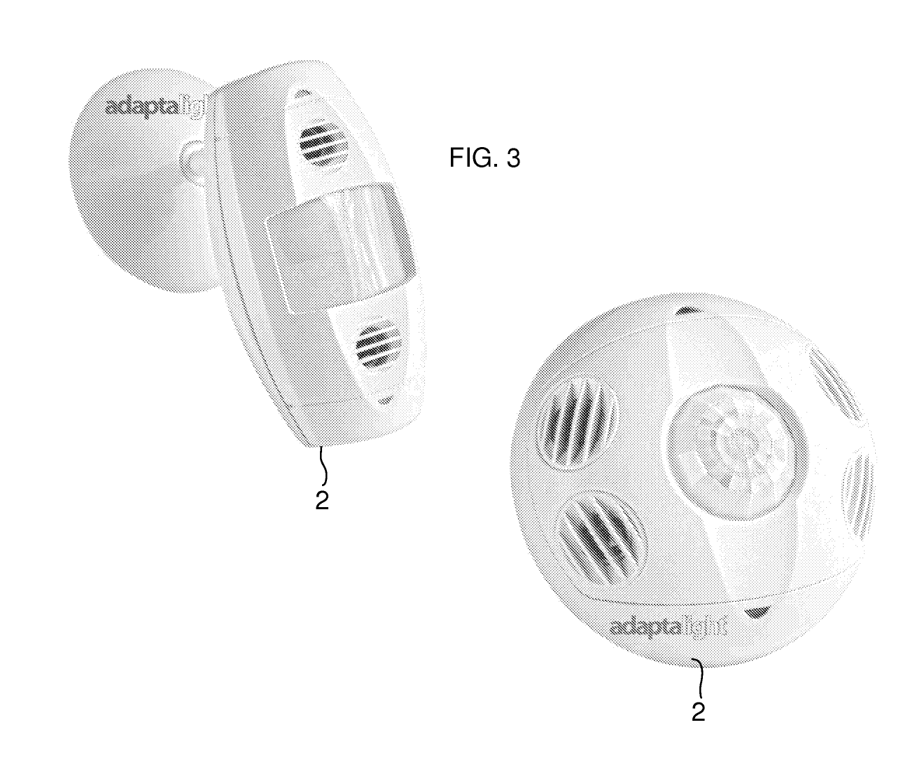

The intensity and Kelvin temperature of light exposure an individual or group experiences may vary from day-to-day, and especially under artificial light conditions may be far from optimal light intensity and Kelvin temperature that humans experience under natural lighting conditions. Scientific studies have shown that individual light therapy whereby an individual must look for hours into a light box with designed light intensity and Kelvin temperature have resulted in benefits to the individual including restoration of normal Circadian rhythm and sleep patterns, among others. The invention is configured to optimize the lighting environment for individuals and groups in terms of light intensity and Kelvin temperature through automated passive methods that do not require conscious participation by the individual such as staring into a light box for two or more hours. Furthermore, beneficial lighting can be delivered passively to large groups of people such as students, elderly residents, prison inmates, and mining camp residents in the far north. The present invention provides an LED light panel 1 or other LED light 1 that is dimmable and Kelvin changeable, various lighting intensity and Kelvin temperature sensors 2 as further shown in FIGS. 2 and 3, and the control system 3 that acquires and processes various sensor and other programmable data to optimally control the LED light intensity and Kelvin temperature in a manner consistent with certain health and human behavior objectives. In other words, the invention does not just provide lighting but provides lighting which changes throughout the day to mimic the changing intensity of natural lighting.

An important aspect of the control system 3 is the ability to define user classes and tasks that can be processed by the control system 3 to determine what control commands should be issued to the light fixtures. User classes in a hospital setting may be such categories as doctor, nurse, therapist, maintenance staff, patient, visitor, etc. whereas each user class could perform a certain number of predefined tasks which in turn would have defined light brightness and Kelvin temperature requirements. For example, in an elder care facility there would be various classes of staff and residents with each class having a subset of task level lighting requirements defined. It is sometimes the case different classes of residents may exist in the same facility including independent living, assisted living, and memory care. Degrees of control of lighting can be defined by user class, for example, with independent living residents enjoying the greatest degree of control and where residents in memory care might have the least degree of control. Automated controls including scheduling might be more widely deployed in the memory care unit relieving the memory care resident from the burden of lighting control and facilitate execution of desirable lighting levels as determined by healthcare professionals.

Desirable lighting levels as determined by healthcare professionals can be automatically achieved by the control system through processing current and anticipated light exposure relative to the desired light exposure profile for individuals and groups and issuing lighting commands to reach the desired profiles.

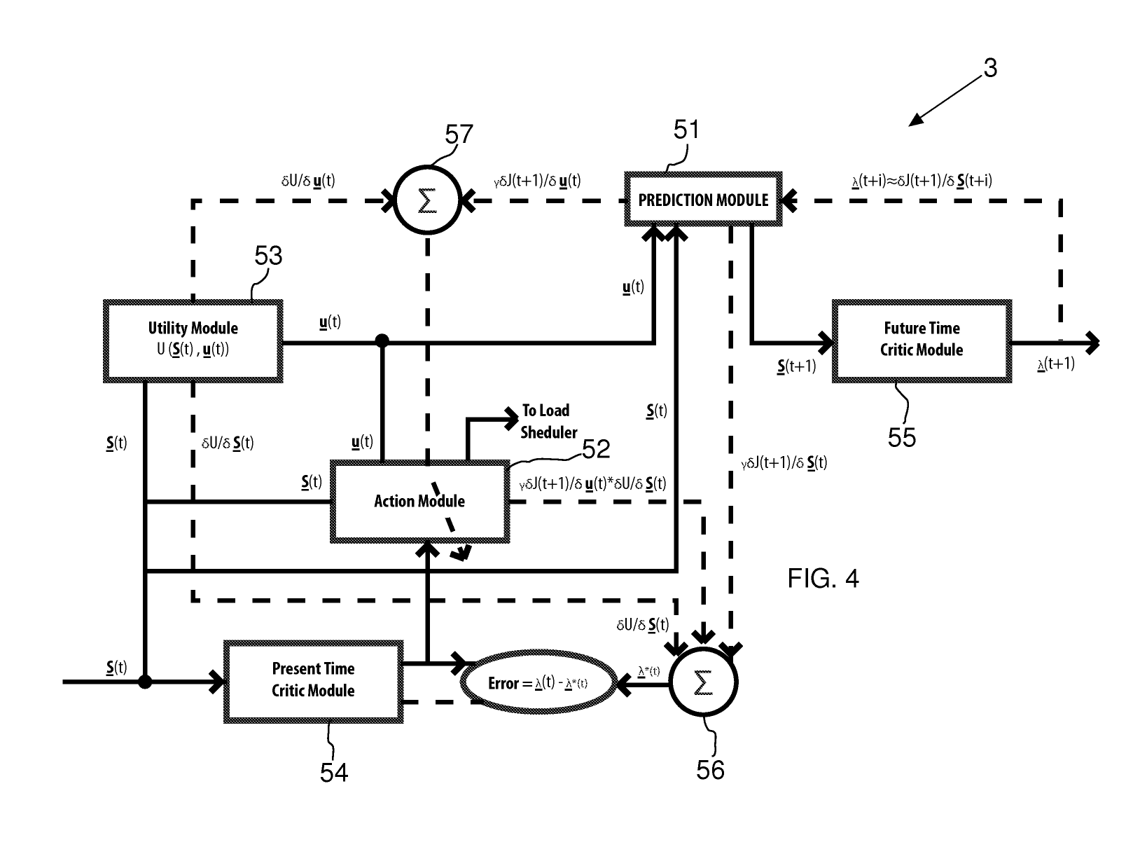

As shown in FIG. 1, the control system 3 has a central control unit 10 for processing the sensor data and a memory unit 11 for storing results and the sensor data. FIGS. 4-6 show further details of the control system 3. More specifically the central control unit 10 may function as a fuzzy neural network processing unit or may be connected to a separate fuzzy neural network processing unit.

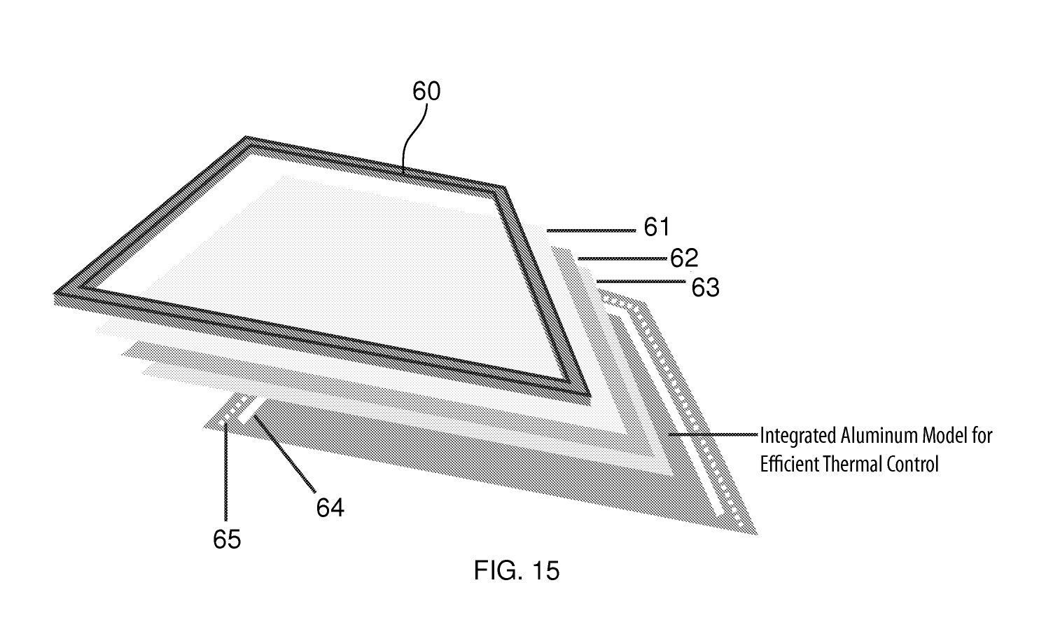

As shown in FIG. 15, the dimmable and Kelvin variable lights of the invention are flat LED panels 60 approximately ⅜ of inch thick in various sizes and shapes, with various LED bulbs, and various LED light fixtures. The LED panel 60 is formed of a diffused lens panel 61, a light guide plate 62, a reflective sheeting 63, a silicon pad 64 with edge-lighting LED 65 on MCPCB. The thin LED panels 60 also offer the benefit of being very low glare devices which allows individuals and groups to be exposed to bright white light of 6,500 Kelvin without being irritated by high glare levels commonly found in other types of bright white light. The LED panels 60 are controlled by a set of wireless sender and receiver units that execute manual commands to change light settings. Furthermore there is the control module 3 residing on a Linux server that includes a scheduling function and can receive and store data from a variety of light sensors as well as external data including weather reports and other sources relevant to probable light conditions. The Linux server also stores lighting profiles for individuals and groups that in combination with the sensor and other input is processed to derive desirable light settings. Generally the desirable light profiles mimic normal sunrise and sunset light conditions helping to assure bright white light is delivered in the morning and through the day, and warm light with less blue light spectrum in the evening. The 6,500 Kelvin light temperature is found in nature and approximates the Kelvin temperature of bright mid-day sunlight. Prior to the deployment of artificial lighting most people were exposed to bright white sunlight of 6,500 Kelvin on a regular basis as they went about their normal daily activities outdoors unless they lived in the far north or south during winter months where the sun may only shine low in the sky for a few hours. As sundown came the Kelvin temperature of the natural light became warmer and this triggered secretion of melatonin by the pineal gland, and suppresses secretion of other chemicals, e.g. cortisol by the adrenal gland in the brain. Melatonin and other secretions help establish normal patterns of alertness and sleep, and also serve to regulate many body functions such as heart rate, blood pressure, and the processing of sugar by the body. Most artificial lighting is at a lower Kelvin temperature than natural sunlight, indeed most interior lighting may only be 3,000 Kelvin. To put the warmness of 3,000 Kelvin lighting into perspective it is important to note that moon light is 4,100 Kelvin, with 3,000 Kelvin being closer to light emitted from a fire. The initial Kelvin variable lights utilized in this invention were fabricated to our specifications of Kelvin variability between 2,700 and 6,500 Kelvin, along with dimming capability in an extremely low glare LED lighting fixture.

The sensors utilized in the invention include a variety of lighting sensors (see FIG. 3) deployed throughout building structures and campuses that are configured to detect ambient light levels and Kelvin temperatures as they change through the day, as well as light sensors 2 that are attached to an individual in the form of a lapel-type pin (FIG. 2) that records the cumulative light intensity and Kelvin temperature exposure the individual experiences through the course of the day. The typical sensor has a data acquisition sensor or layer 21, a data processor and memory layer 22, and a wireless transmitter or layer 23.

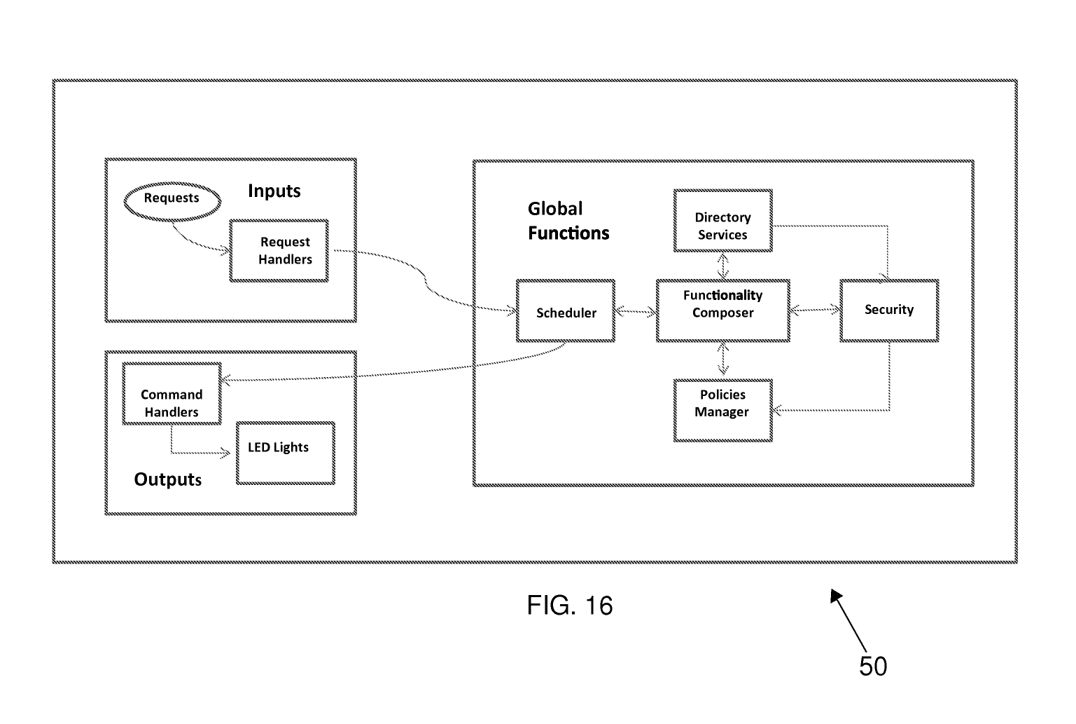

The building-level sensor data is useful in determining the lighting exposure of groups of people, e.g. a group of students in a school, a group of workers in a mining camp in the far north, or a group of elderly in an old age facility. The building-level data can be transmitted to a building lighting control system 50 (see FIG. 16) to make adjustments in the LED light intensity and Kelvin temperature, and the individual lighting exposure data also provides input and can be balanced by the intelligent control system to optimize lighting levels for the group as well as individuals in the group.

The individual-level light sensor 2 can precisely record the light intensity and Kelvin temperature the individual 8 has experienced over a period of time. By itself this type of individual light exposure data has been analyzed by scientists to prescribe light-box and other similar dedicated light therapy, e.g. specialized goggles, and has been described in peer-reviewed literature. Therefore the cumulative light exposure of an individual is recorded and lighting is adjusted throughout the day in dependence on the time and previous light exposure.

One embodiment of the invention can utilize light data gathered by individual-level light sensors 2 in the form of a pin, wrist band, identity card or other wearable device to provide real-time input into the LED lighting control system 3, 50 that will then make real-time changes to light intensity and Kelvin temperature in order to optimize the individual's light exposure in an effort to meet certain light exposure objectives consistent with other medical or work objectives (see FIGS. 2, 4, 5, 6). The individual-level light data can be received and processed by the control system 3, 50 in a real-time manner along with the building-level data to be processed by the control system 3, 50 to optimize lighting intensity and Kelvin temperature for the individual, group, or the individual within a group. The lapel-type light intensity and Kelvin temperature data gathering pin 2 can be easily worn by the individual and is configured to both gather data and through wireless radio frequency telecommunication supply that data to the control system 3. A data gathering pin currently exists, however the invention additionally transmits data gathered by the wearable device to the control system 3 for real-time processing.

FIG. 4 shows one embodiment of the control system 3. The control system 3 is formed of various software derived modules. Input data is received by a utility module 53 and a present time critical module 54. The data from modules 53 and 54 is forwarded to an action module 52 and/or a prediction module 51 which predicts current lighting needs and forwards this data to a future time critical module 55. Summing units 56 and 57 are provided for combining data. In essence the control system 3 receives the sensor data from the individual and building sensors and combines this data with known individual needs and situational needs of events to occur in the near term for the individuals.

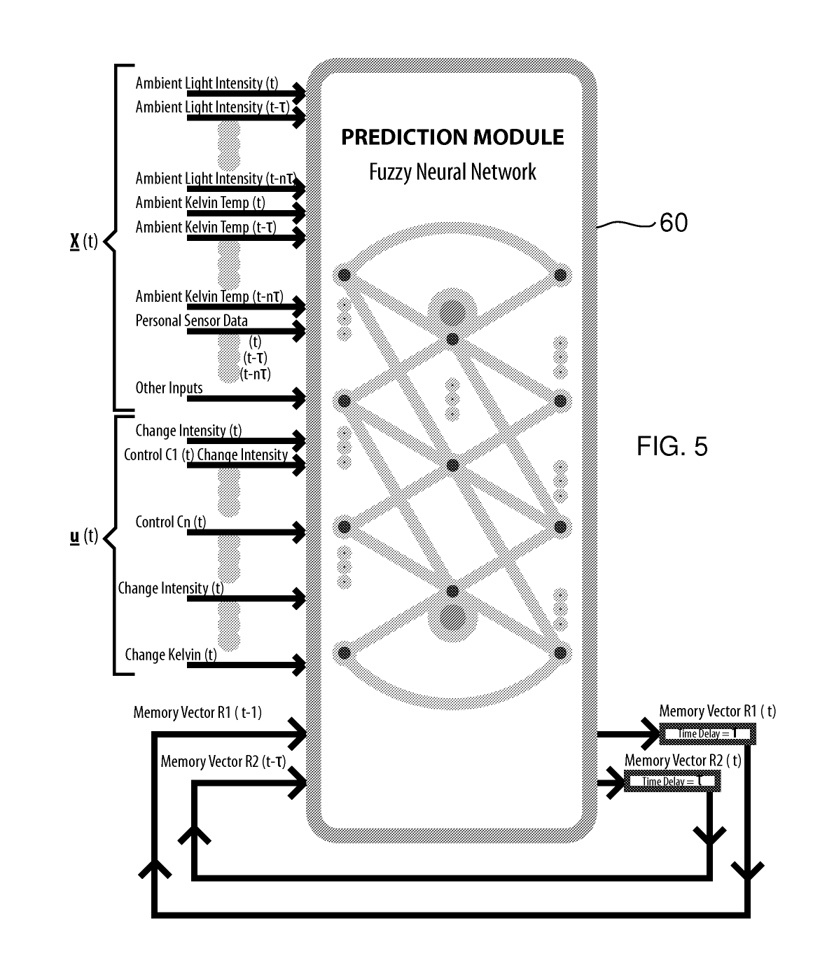

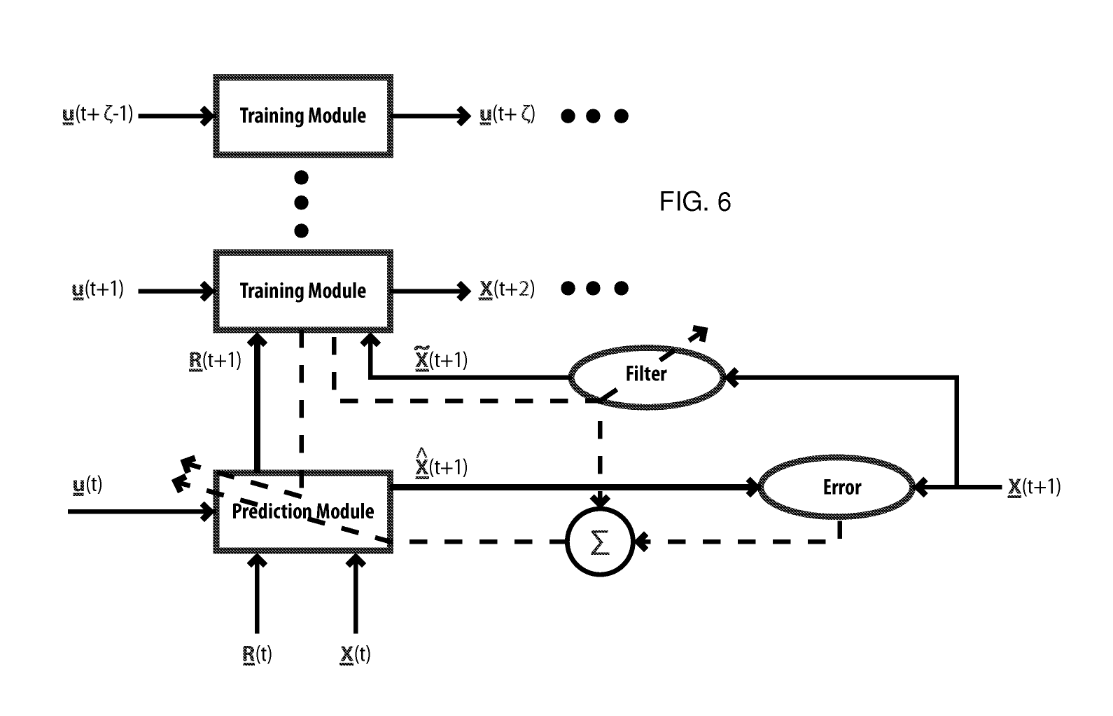

The control system 3, 50 of the invention exists at several hierarchical levels that can operate individually and a semi-autonomous mode, or collectively through the application of fuzzy neural networks (see FIGS. 5 and 6) that are part of the control system 3 and specifically part of the invention to optimize light intensity and Kelvin temperature exposure for the individual, group, or individual within a group. The first two manners of control: the individual and the group are relatively intuitive and are described below. It is the third level of control, the individual in the group that may be less intuitive and is uniquely part of the invention.

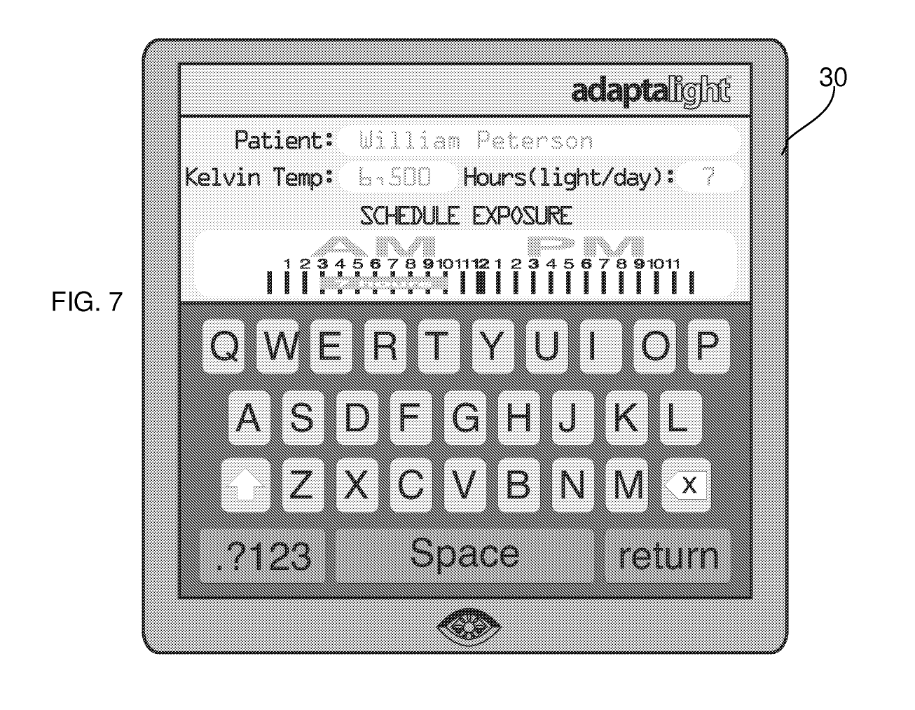

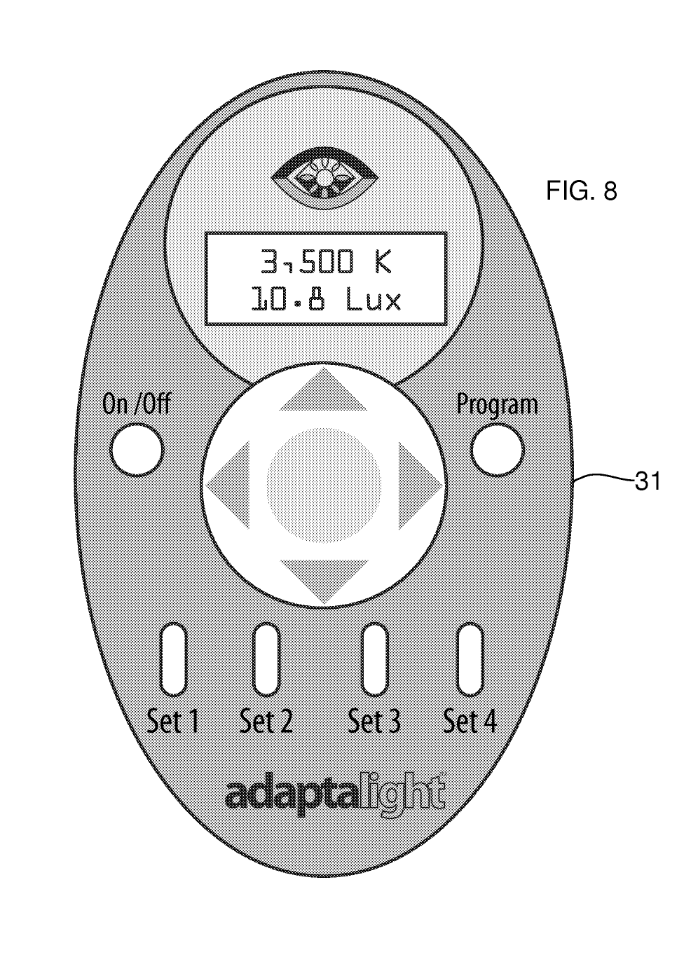

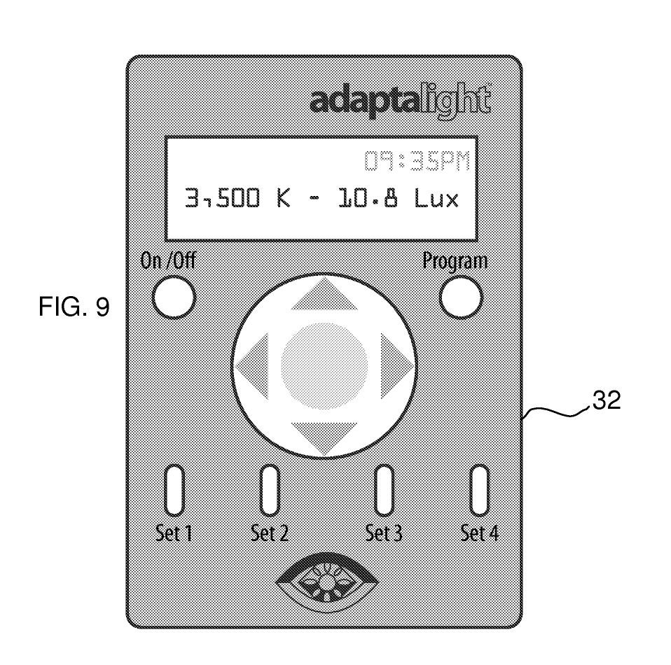

Another embodiment of this invention optimizes light intensity and Kelvin temperature for an individual by including in the automatic analysis performed by the control system health objectives entered into a programmable controller 30 (FIG. 7) as determined by medical professionals in terms of optimal light exposure, the data gathered by building-level and/or individual-level light sensors 2, an automated prediction of likely lighting exposure in the near-term (i.e. that “day”), and issues control commands to the LED light fixture to adjust its brightness and Kelvin temperature in order to optimize light exposure for the individual on a daily cyclical basis (see FIG. 4). The lighting control can also be performed manually by the individual via a hand held controller 31 or other supervisory individual via a wall controller 32 (see FIGS. 8 and 9). It is important to note that the invention processes historical lighting data and from real-time sensor devices 2, and also projects lighting exposure in the near-term along with likely duration of exposure to determine what light intensity and Kelvin temperature settings would best achieve daily light exposure objectives taking into account near future ambient light predictions that may be generated by weather reports and the like.

In another embodiment, in a far north mining camp during the winter, natural light exposure to high Kelvin temperature light, that is greater than 4,000 Kelvin, is extremely limited and the control system would project very little light exposure for the balance of the day, and might provide bright white exposure for the period of time the individual is exposed to the LED light. Conversely, at the same mining camp in summer, the control system would project that the individual would likely be exposed to high levels of natural sunlight throughout the day (if working outdoors) and would not expose the individual to high Kelvin light. Similarly, shift workers at the mining camp may not be exposed to natural light any time of the year that promotes normal circadian rhythm sleep patterns. Through effective control of light intensity and Kelvin temperature afforded by this invention the normal circadian rhythms of shift workers can be promoted through achievement of lighting goals established by medical professionals.

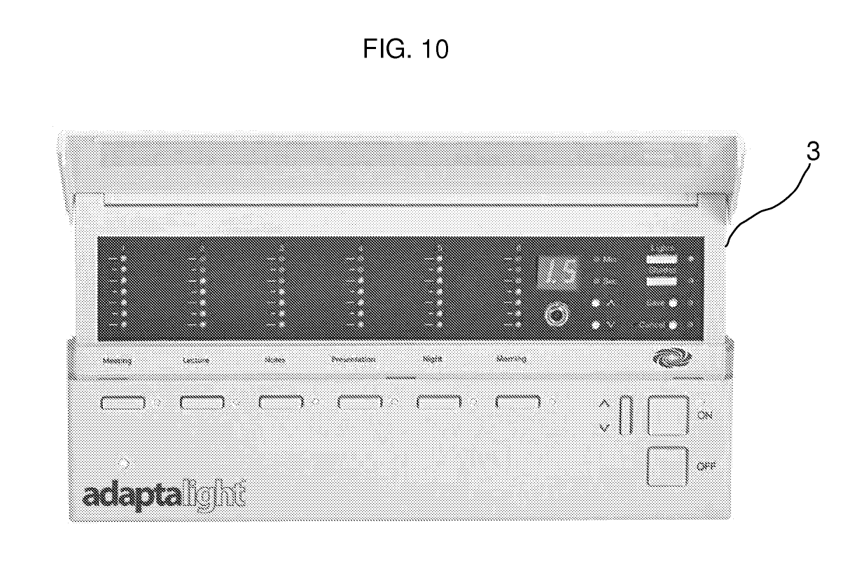

In another embodiment, residents in old age facilities (FIG. 10) often do not receive a medically established desirable level of bright high-Kelvin light normally obtained through exposure to natural sunlight. Many elderly patients have disrupted sleep patterns and are prescribed medications or active light therapy in an attempt to promote normal healthy sleep. The invention can deliver the bright high-Kelvin temperature light recommended by healthcare professionals in a passive manner that does not require the patient to perform any tasks or take any medications to promote sleep. Currently, light therapy consisting of exposure to bright high-Kelvin light is achieved through requiring the patient to stare into a light box or other device for some two hours. Furthermore, such light therapy does not take into account the amount of light the patient has been, and likely will be, exposed to during the day. For example, if it is a bright sunny day and a comfortable temperature, the patient may have an opportunity to be exposed to desirable natural light, and the data gathering pin or sensor 2 would record that data and communicate it to the lighting control system 3. When the patient returns to their room on a sunny day when they were exposed to natural light the control system would include in its analysis that light exposure and not seek to over-expose the patient to high-Kelvin light. More likely the patient on a normal day has not been exposed to desirable levels of natural light, e.g. on a rainy day or a day the elder has not been exposed to enough natural light the control system would compensate for the anticipated lack of exposure by providing high Kelvin temperature lighting indoors.

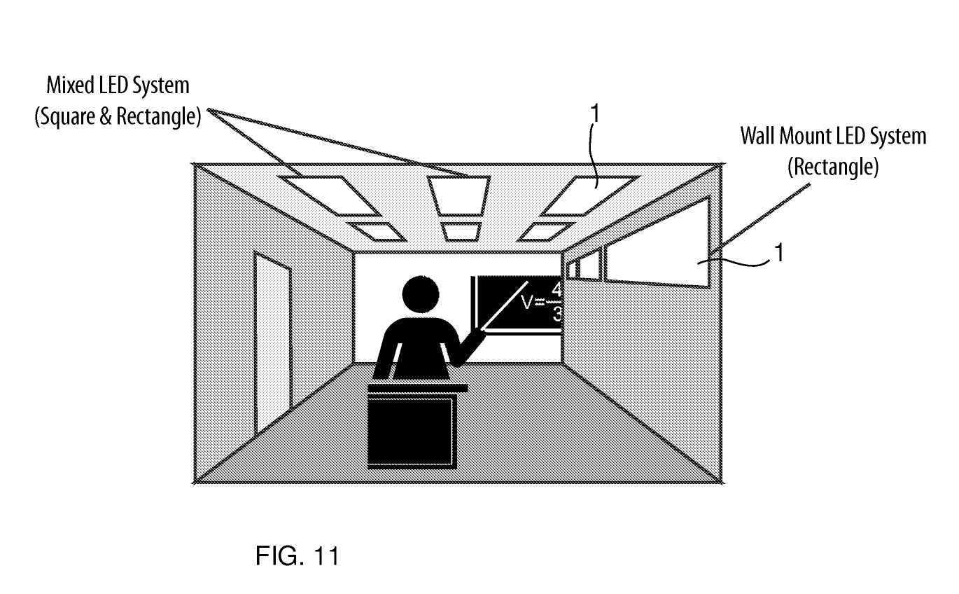

In another embodiment, lighting control for groups of people would be accomplished similarly through processing light exposure data acquired from a variety of sensors placed in key areas indoors and outdoors (FIG. 3), analyzing this data along with other inputs such as upcoming activities, and issuing control commands to the LED lighting designed to optimize light intensity and Kelvin temperature exposure. For example, a group of third grade students (FIG. 11) might be scheduled to take a standardized test after lunch so the control system would cause the LED lighting to emit high intensity and high Kelvin temperature light consistent with peer-reviewed scientific studies (Mott) that indicate elevated test scores when students have been exposed to high intensity high Kelvin temperature immediately before and during standardized tests. Conversely, if the same third grade class was to have nap/rest time, the control system would dim the lights and change the Kelvin temperature to 2,500 Kelvin so as to promote a restful environment.

In another embodiment of this invention, group lighting control in an elder care facility where most patients receive little or no bright sunlight can be implemented through the control system by programming it to be aware of the daily schedule (FIG. 7). In this example the control system would cause the LED lighting in the resident's and common rooms to provide bright 6,500 Kelvin light during the day, and 2,700 Kelvin light during the evening, thus promoting lighting intensity and Kelvin temperature that has been determined by medical experts to be optimal with respect to melatonin release by the pineal gland in the brain which is essential to a normal circadian rhythm and sleep cycle. Experiments conducted at the Rennselaer Polytechnic Institute's Lighting Research Center Light and Health Institute have determined the levels of light intensity and Kelvin temperature required to entrain circadian rhythm and we have designed our LED light and control systems to achieve the required levels of light. In both the school and the elderly care facility, the lighting can be controlled by authorized individuals as well as through automated control.

In another embodiment, lighting control that balances the individual's needs along with the groups needs is accomplished through advanced artificial intelligence control that has the ability to balance individual and group lighting exposure objectives (FIGS. 5 and 6). The basis for the artificial intelligence control mechanism is fuzzy neural networks 60 that seek to identify optimal behavioral control across a number of weighted variables. Fuzzy sets mathematically differ greatly from Boolean sets in that with Boolean sets membership in a set is absolute and can be represented in a control sense as a zero or a one. Fuzzy sets allow for a membership value in a set. A simple example is the set of tall people that we can arbitrarily define as anyone six feet tall or taller. The “tall” people would be assigned a one and all others a zero. This implies that someone 5 feet 11.999 inches would receive a zero and someone 0.001 of an inch taller a one when there is virtually no difference in their height. From a control perspective Boolean sets can be blunt tools. When the same group of people is treated with fuzzy sets the individual only 0.001 of an inch shorter than the six foot individual would have nearly the same membership value in the set of tall people. Fuzzy sets offer a far sharper tool from a control perspective, and when combined with a pattern detecting neural network are used to optimize light settings for an individual in a group. There is currently no other LED lighting control system with the capability to optimize Kelvin temperature and intensity light settings for an individual within a group.

For example, an individual that has been diagnosed with a sleeping disorder might be given a higher weight in a group than an individual in the same group who has no sleep disorder. When the artificial intelligent processor of the invention analyzes the various building and individual sensor data, it will take into account the membership values (FIG. 5 “other input”) of the individuals in the group in terms of their sensitivity to lighting conditions, and when optimizing lighting for the group will weight those lighting-sensitive individuals in the group higher, the optimization algorithm will result in controlling the lighting in a way that optimizes both the individual and group lighting exposure. If the individuals in the group are equipped with our light gathering data pin 2 (FIG. 2), the lighting exposure during group activities will be recorded and used as input to control lighting intensity and Kelvin temperature exposure in personal spaces. Thus the lighting needs of both the group and the individual can be optimized through multi-level intelligent control that is a central part of this invention.

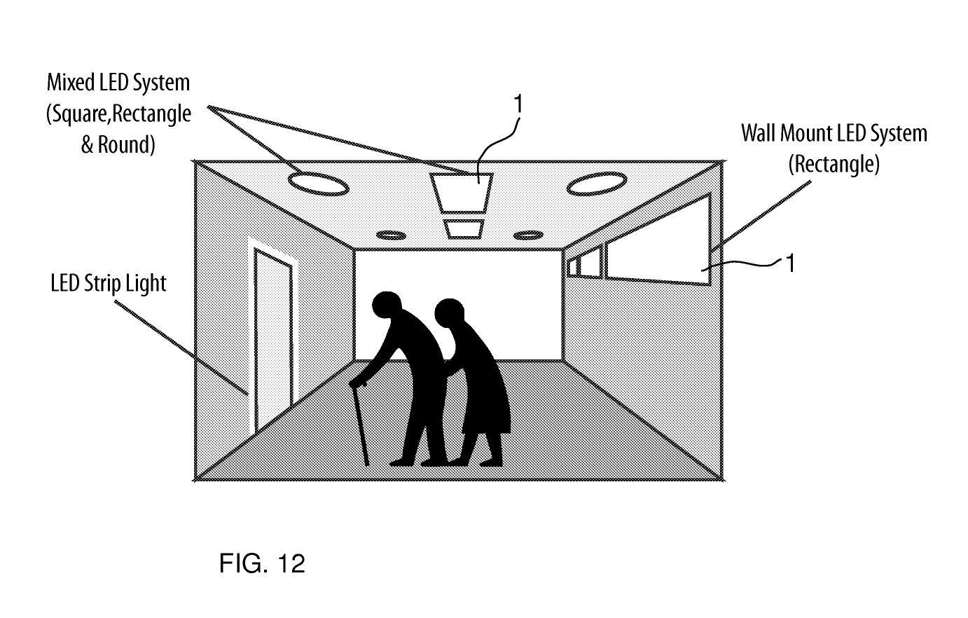

This invention will utilize a number of different types of pre-existing lighting sensors that are commonly available that measure ambient light conditions in a variety of facilities (FIG. 3). They are currently used to provide data to control systems to raise or lower lighting levels, raise or lower window blinds, and other similar tasks (FIG. 12). These systems do not take into account real-time light exposure of individuals, nor do the control systems seek to optimize lighting conditions at multiple levels of individual or group requirements. Light data gathering sensors and basic control systems are currently available from Crestron, Lutron, EuControls, and other notable vendors. Additionally, a light intensity and Kelvin temperature data gathering pin has been developed by Rensselaer Polytechnic Institute (RPI). The invention includes utilizing the data gathering aspects of this or similar light data gathering pin and adding wireless radio frequency communication capability to it so real-time data can be transmitted to our control system module. It is the unique data gathering, processing, and analysis aspects of our invention that extend the field of lighting control into new capabilities that can optimize lighting intensity and Kelvin temperature across individuals and groups based on real-time data acquisition, transmission, processing, and prediction through our fuzzy neural network module (FIGS. 4-6).

An embodiment of the present system provides an LED light panel that has preprogrammed light settings typically required by different groups or individuals (FIG. 7). This lower cost approach can avail lighting Kelvin temperature control for budget-constrained situations or in environments where regularly scheduled activities are predictable and able to be addressed through less sophisticated lighting control than described above. These pre-programmed light intensity and Kelvin temperature control settings are based on scientific research and can be effectively deployed in both institutional and consumer products to provide desirable levels of light intensity and Kelvin temperature for specific activities.

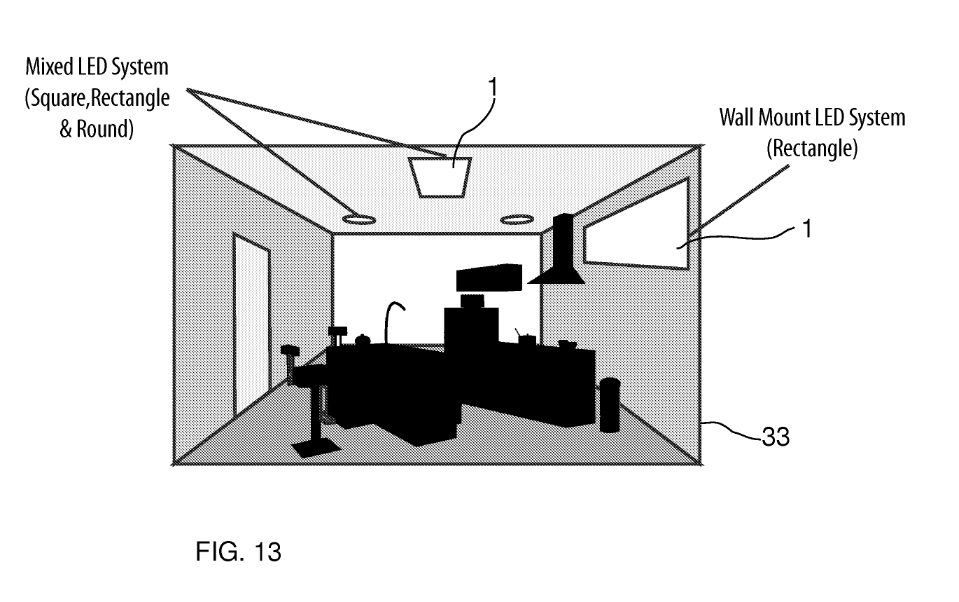

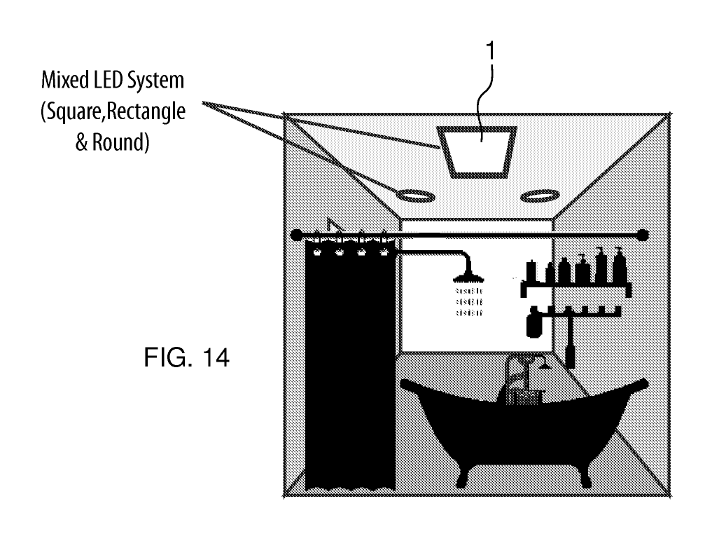

Another embodiment of the lower cost LED light intensity and Kelvin temperature controlled light is a consumer product for use in a kitchen 33, FIG. 13, that has preprogrammed light settings for specific activities (FIGS. 8 and 9). For example, in food preparation and cleaning activities high intensity and high-Kelvin temperature light is desired for safety and cleanliness reasons, and lower intensity and warmer Kelvin temperature light is desirable for dining activities. Our kitchen light is pre-programmed to deliver various desirable light settings with minimal action required on the part of the consumer (FIG. 13). Similarly, our bathroom light (FIG. 14) is preprogrammed for makeup application settings utilizing optimal high light intensity levels and Kelvin temperature settings that promote optimal light conditions for different skin tones while also providing lower intensity warmer light for normal bathing activities.

1. An LED lighting unit that can be adjusted for both brightness and Kelvin temperature, the LED lighting unit comprising:
LED lights for outputting light;
a power switch controlling power to said LED lights for turning said LED lights on and off;
a light dimming switch for adjusting a power output of said LED lights;
a Kelvin temperature changing switch for controlling the Kelvin temperature of said LED lights;
a control unit controlling individual and groups of said lights controlled by said power switch, said control unit having a memory;
first sensors to be worn by individuals for gathering individual light data including lighting intensity data and Kelvin temperature data experienced by the individuals, said first sensors transmitting the individual light data to said control unit;
second sensors to be disposed in a building for collecting building light data including light intensity building data and Kelvin temperature building data and transmitting the building light data to said control unit;
said memory unit of said control unit storing the individual light data and the building light data, along with desired data including desired individual data and desired group data associated with desired light intensity and desired Kelvin temperature;
a fuzzy neural network processing unit having data inputs and determining optimal light exposure for an individual, a group, or an individual within the group based on at least one of the individual light data, the building light data or the desired data, and sending at least one output signal to said control unit; and
said control unit receiving the output signal and operating said power switch, said light dimming switch and said Kelvin temperature changing switch based on the output signal and the desired light intensity and the desired Kelvin temperature for the individual and/or the group.

2. The LED lighting unit according to claim 1, wherein said first and second sensors have wireless transmitters for transmitting the individual light data and the building light data.

3. The LED lighting unit according to claim 1, wherein said control unit receives a command from an authorized individual to change light intensity and/or the Kelvin temperature of the light.

4. The LED lighting unit according to claim 2, wherein said control unit processes at least one of the individual light data, the building light data or the desired data and sends the control signal to change light intensity and the Kelvin temperature of said LED lights to optimize emitted light for the individual.

5. The LED lighting unit according to claim 2, wherein said control unit processes at least one of the individual light data, the building light data or the desired data and sends the control signal to change light intensity and the Kelvin temperature of said LED lights to optimizing emitted light for the group.

6. The LED lighting unit according to claim 5, wherein said fuzzy neural network processing unit introduces weightings for optimizing individual light exposure needs within the group in such a way that the individuals in the group most in need of light intensity and Kelvin temperature optimization are given a greater weight within the group in determining an optimal light intensity and the Kelvin temperature for the group.

7. The LED lighting unit according to claim 6, wherein said memory unit stores the individual light data for the group, the individual light data is utilized by said control unit to determine the optimal light intensity and the Kelvin temperature settings for the individual at a later point in time.

8. The LED lighting unit according to claim 5, wherein activity data stored in said memory unit relating to planned group activity is utilized in advance of a particular activity by said control unit to optimize the light intensity and the Kelvin temperature for the group in such a way as to deliver light consistent with scientific studies that indicate that group behavior is influenced in a desired manner when the group is exposed to specific levels of the light intensity and the Kelvin temperature.

9. The LED lighting unit according to claim 4, wherein activity data stored in said memory unit relating to planned or desired individual activity is utilized in advance of a particular activity by said control unit to optimize the light intensity and the Kelvin temperature for the individual in such a way as to deliver light consistent with scientific studies that indicate that individual behavior is influenced in a desired manner when the individual is exposed to specific levels of the light intensity and the Kelvin temperature.

10. The LED lighting unit according to claim 1, wherein said control unit stores programmable light intensity and Kelvin temperature information.

11. The LED lighting unit according to claim 1, further comprising a hand controller for communicating with said control unit for setting a new light intensity and a new Kelvin temperature.

12. A control unit for coupling with a memory unit and a fuzzy neural network processor, the control unit comprising:
a prediction module receiving light data and generating a control value based on the light data; and
an action module coupled to said prediction module, said action module generating an output value for controlling operations of a switch, a dimmer, and a Kelvin changing element.

13. The control unit according to claim 12, wherein the light data is one of actual light data, estimated light data, or specified light exposure data.

14. The control unit according to claim 12, wherein said prediction module receives the light data and uses the fuzzy neural network processor to combine the light data with individual and group light data to generate the control value.

15. The control unit according to claim 12, wherein said prediction module receives environmental information and utilizes the fuzzy neural network processor to combine the environmental information and the light data to generate the control value.

16. A method for controlling light exposure of an individual, which comprises the steps of:
providing a control unit controlling individual and groups of lights controlled by a power switch, the control unit having a memory unit;
providing a first sensor being worn by the individual for gathering individual light data including lighting intensity data and Kelvin temperature data experienced by the individual, the first sensor transmitting the individual light data to the control unit;
providing second sensors disposing in a building used by the individual for collecting building light data including light intensity building data and Kelvin temperature building data emitted in the building and transmitting the building light data to the control unit;
storing in the memory unit of the control unit the individual light data and the building light data, along with desired data including desired light intensity and desired Kelvin temperature;
determining an optimal light exposure for the individual or the individual within a group based on at least one of the individual light data, the building light data or the desired data, and generating at least one output signal based on the optimal light exposure;
sending the least one output signal to the control unit; and
operating at least one of the power switch, a light dimming switch or a Kelvin temperature changing switch controlling the lights based on the output signal to produce a overall light intensity and Kelvin temperature pattern for the individual.

17. The method according to claim 16, which further comprises utilizing a fuzzy neural network processing unit for determining the optimal light exposure for the individual based on at least one of the individual light data, the building light data or the desired data.

18. The method according to claim 16, which further comprises providing, via the individual, new desired data to the control unit for changing a light intensity and/or Kelvin temperature of the light.

19. The method according to claim 17, which further comprises weighting inputs to the fuzzy neural network processing unit for optimizing light exposure needs of the individual within the group such that the individual in the group most in need of light intensity and Kelvin temperature optimization is given a greater weighting within the group in determining an optimal light intensity and the Kelvin temperature for the group.

20. The method according to claim 17, which further comprises utilizing activity data stored in the memory unit of planned activities in advance of a particular activity by the control unit to optimize the light intensity and the Kelvin temperature for the individual in such a way as to deliver light consistent with scientific studies that indicate that behavior is influenced in a desired manner when the individual is exposed to specific levels of light intensity and the Kelvin temperature.

21. The method according to claim 17, which further comprises:
controlling a quantity of the light on a daily basis based on a 24 hour circadian rhythm; and
controlling the light based on the circadian rhythm at least once per hour.

22. The method according to claim 20, which further comprises selecting the planned activity from the group consisting of sleep patterns, testing taking, activities performed in mornings and activities performed in evening hours.

23. The method according to claim 20, wherein the individual light data includes environmental light received by the individual being exposed to natural sun light.

24. A method for controlling light exposure of individuals within groups of individuals, which comprises the steps of:
providing a control unit controlling individual and groups of lights controlled by a power switch, the control unit having a memory unit;
providing first sensors being worn by the individuals for gathering individual light data including lighting intensity data and Kelvin temperature data experienced by each of the individuals, the first sensors transmitting the individual light data to the control unit;
providing second sensors disposing in a building used by the individuals for collecting building light data including light intensity building data and Kelvin temperature building data emitted in the building and transmitting the building light data to the control unit;
storing in the memory unit of the control unit the individual light data and the building light data, along with desired data including desired light intensity and desired Kelvin temperature;
determining an optimal light exposure for the individuals based on at least one of the individual light data, the building light data or the desired data, and generating at least one output signal based on the optimal light exposure;
sending the least one output signal to the control unit; and
operating at least one of the power switch, a light dimming switch or a Kelvin temperature changing switch controlling the lights based on the output signal to produce a overall light intensity and Kelvin temperature pattern for the individuals.

25. The method according to claim 24, which further comprises weighting the desired data for an individual within the groups such that the individual in a group most in need of light intensity and Kelvin temperature optimization is given a greater weighting within the group in determining an optimal light intensity and the Kelvin temperature for the group.

26. The method according to claim 24, which further comprises exposing each of the groups to different light intensity and Kelvin temperature patterns within different areas of the building.
